(19) United States
(12) Patent Application Publication
(10) Pub. No.: US 2026/0151075 A1
(43) Pub. Date: Jun. 4, 2026

(54) SYSTEM AND METHOD FOR REMOTE SCREENING AND DIAGNOSIS OF CONTACT ALLERGIES

(71) Applicant: THE BOARD OF TRUSTEES OF THE LELAND STANFORD JUNIOR UNIVERSITY, Stanford, CA (US)

(72) Inventors: Golara Honari, Menlo Park, CA (US); John E. Paderi, San Francisco, CA (US); Serena Yeung, Stanford, CA (US)

(21) Appl. No.: 19/126,611

(22) PCT Filed: Nov. 7, 2023
(86) PCT No.: PCT/US2023/036938

§ 371 Date: May 1, 2025

Related U.S. Application Data
(60) Provisional application No. 63/423,584, filed on Nov. 8, 2022.

(51) Int. Cl. A61B 5/00; A61B 10/00

(52) U.S. Cl. CPC A61B 5/411; A61B 5/0013; A61B 5/0022; A61B 5/0077; A61B 5/7267; A61B 5/7275; A61B 10/0035

(57) ABSTRACT

Methods and compositions are provided that enable users to interact with a multimodal digital health platform to assess inflammatory skin conditions, including eczema. Screening for contact allergies is performed with a transdermal adhesive device to deliver contact allergens for epicutaneous patch tests, herein termed a “Contact Allergen Panel”. Automated detection and interpretation may use machine learning based on image feature extraction and integration with allergen skin-sensitizing potency and significance to provide an analysis of reactivity to an allergen of interest.

### CROSS REFERENCE TO OTHER APPLICATIONS

This application claims the benefit of U.S. Provisional Application No. 63/423,584, filed Nov. 8, 2022, the contents of which are hereby incorporated by reference in its entirety.

### BACKGROUND

Eczematous dermatoses are a group of common inflammatory skin disorders, also known as dermatitis Atopic dermatitis (AD) and contact dermatitis (CD) are the most common types of eczema. Eczema negatively impacts the quality of life and has a high economic burden. Atopic dermatitis (AD) is the most common chronic inflammatory skin disease. AD is estimated to affect about 13% of the pediatric and 7% of the adult population in the United States US. Contact dermatitis (CD), including allergic contact dermatitis and irritant contact dermatitis, are among the most common types of eczema. An estimated direct cost of care associated with contact dermatitis exceeds $1.5 billion annually. Allergic contact dermatitis commonly affects the general population and individuals with atopic dermatitis. The gold standard for diagnosis of contact allergy is epicutaneous patch testing, which is an in vivo test procedure to confirm T-lymphocyte-mediated allergic diseases. The term “patch test” comes from the historical fact that initially, patches of fabric or paper were soaked with the test substance and applied to the skin.

To identify the causes of contact dermatitis, in the epicutaneous patch test procedure, a substance suspected to cause contact allergy or irritation is applied to normal skin under occlusion for a certain period of time, and subsequently, the reactions on the skin are monitored for a few days after removal of the test substance to identify an inflammatory response to the suspected substance. The concentration and formulation of the test substance should be controlled to avoid sensitization, irritation, or false negative reactions. Over the past decades, a few devices and patches have been used to carry on patch testing.

Patch testing helps identify common causes of allergic contact dermatitis, guides the management of individuals with chronic eczema, and improves quality of life. A majority of patients with chronic eczema, including patients with AD, may benefit from patch testing. Patch testing helps improve quality of life and cost of care in patients with chronic eczema.

Contact allergies are mediated through a delayed-type immune reaction (Gel and Coombs type IV hypersensitivity reaction). The standard of care for assessment of contact allergies requires transcutaneous exposure to a low concentration of the tested substance (allergen) for two days, followed by removal of the allergens from the skin and examination of the test site reactions within 4-7 days after allergen application.

The are problems with the current standard of care. While patch testing is a non-invasive, somewhat simple procedure, the allergen selection, application, and interpretation of the results requires expertise. Currently, patch testing is only offered in specialty clinics, and the procedure typically requires three visits to the specialty clinic within a week. The limited number of specialty clinics nationwide and the fact that specialty clinics are mostly concentrated in large cities and academic centers make access to care very limited. Only a small portion of patients who benefit from patch testing have access to this test.

Currently, test panels are not waterproof; therefore, patients are asked to refrain from bathing during the two days of allergen application. In addition, skin markers are typically used by the clinic staff to draw on the skin and mark the location of the applied allergens. These markings can easily be washed off; therefore, patients are advised to continue to refrain from washing the skin of the tested area until the final assessment (additional 2-5 days after removal of the allergens), which leads to a total of four to seven days. A commute to a specialty clinic 2-3 times within a week, and bathing restrictions pose significant inconveniences for patients who might benefit from patch testing. Also, a limited number of specialty clinics lead to long wait times and delaying care for many patients.

Additionally, currently, patch test procedures are typically performed by one of two methods; 1) hand loading a small amount of the test allergen in a small chamber made of aluminum or other materials; often multiple chambers are affixed to an adhesive tape and applied to the skin or 2) by using a preloaded set of allergen panels called Thin Layer Rapid Use test (TRUE test) in which the allergens are incorporated in a thin layer of a thin flexible solid vehicle and applied on the skin. Both these methods have disadvantages. The first method is prone to human error in the loading of the allergen chambers that could present either as wrong allergen or inaccurate dose. While TRUE test is advantageous in delivering a consistent dose of the allergens, the adhesive tapes are not waterproof.

Improved methods for determining causes inflammatory skin conditions are of great clinical interest; and are addressed by the disclosure herein.

### SUMMARY OF THE DESCRIPTION

A system and method are provided for remote assessment of delayed-type contact allergies. The methods and compositions of the disclosure enable users to interact with a multimodal digital health platform to assess inflammatory skin conditions, including eczema. Screening for contact allergies is performed with a transdermal adhesive device to deliver contact allergens for epicutaneous patch tests, herein termed a “Contact Allergen Panel”. The borders of each allergen panel may comprise a unique multi-colored pattern that specifies the location of each allergen, and provides a point of reference for monitoring the skin reactions on each site after allergens are removed from the skin surface. The multicolored markings also provide color calibration tools to normalize skin reactions according to skin tone. A trained convolutional neural network makes automated detection and interpretation of skin reactions to allergens using digital images.

There is a need to improve access and minimize inconveniences associated with the current practice; and the disclosure herein provides a method to facilitate “patch testing” with an improved test kit that facilitates use by the subject. The test kit includes an optional platform that allows testing in a remote setting, such as in a home or a primary care facility, rather than in a specialty clinic. The test kit may include one or more of a device, hardware, software, algorithms, and communication tools.

Improvements include, without limitation, accurate location, dose, and uniform delivery of each allergen, as well as additional features to provide water resistance, feasible marking, and multi-colored ornamentation to facilitate interpretation based on images.

The device, a Contact Allergen Panel, can include components that facilitate the application of one or more allergens to the skin of a subject in need thereof. The device components can include one or more of a multilayer adhesive test panel, including a covering that may be waterproof or water-resistant, allergens, and one or more carriers for an allergen. The test kit may also include markings that facilitate one or more components to assist information capture (such as a digital photograph), the communication of such information, and interpretation of the information.

Several embodiments disclosed herein relate to systems, devices, and methods to facilitate remote assessment of inflammatory skin disorders and, more specifically, relate to systems, devices, and methods to assist with the screening of allergic contact dermatitis by allowing “patch testing” to be done remotely.

In an embodiments, a device to facilitate transcutaneous application of contact allergens is provided. In preferred embodiments, the device allows for remote application, monitoring, and communication of information to a healthcare provider. One or more of the following components may be included in embodiments of the invention: 1) a patch testing device; 2) a secure internet interface for communications between patients and providers, 3) a system for collecting and analyzing clinical data enhanced by machine learning; 4) a system for capturing clinical images that allow normalization of captured images by color and size in different skin tones; 5) a device to carry allergens for patch test; 6) interpretation of patch test results enhanced by machine learning; 7) a platform for communicating the results of patch test and management options to the patient. One or more of the above components may be used in various embodiments of this invention. In some embodiments, the test kit includes a digital health platform, which may include software, hardware, and communication tools.

The Contact Allergen Panel device may comprise components: 1) a flexible water-resistant or waterproof layer; 2) a skin-contacting adhesive; 3) one or a plurality of allergens; and optionally 4) an empty chamber to be used for customized allergens per an individual's needs. Additional device features to facilitate use may include 5) a color normalization component; 6) an allergen identification marker; 7) an allergen-carrying component; 8) separation or barrier to prevent cross-contamination of allergens; 9) an identification marker, such as for serial number and patient identification; 10) external packaging. These components serve to deliver specific allergens to the skin for transcutaneous absorption while preventing cross-contamination. Additional features provide for color calibration and allergen identification to facilitate the interpretation of patch test reactions in different skin tones. The optional waterproof or water-resistant cover feature allows bathing while the patch kit is applied to the skin.

In some embodiments, a digital platform for collecting patient data is utilized in combination with methods for collecting relevant clinical data from patients with inflammatory skin disease, eczema, and putative contact allergies. Clinical images from involved areas of skin (skin eruption), combined with the patient-provided clinical history, subjective complaints, and environmental exposures, including personal skin care product ingredient lists, medications, and occupational exposures, are uploaded into an interactive digital health platform compliant with Health Insurance Portability and Accountability Act (HIPPA), for securely communicating information between the patient and a healthcare provider (HCP). The digital health platform is readily available via internet and can be accessed by users using smartphones, tablets, computers, or the like. Data will be transmitted to a central server via internet. The digital health platform can also be used in the transmittal of patch test results.

In some embodiments a method is provided for automating data interpretation. Automated detection and interpretation may use trained convolutional neural networks based on image feature extraction and integration with allergen skin-sensitizing potency and significance-prevalence index number (a composite measure of allergen prevalence and clinical relevance) to arrive at a probability distribution over International Contact Dermatitis Research Group scores. In some embodiments, a clinician's input is also included.

### BRIEF DESCRIPTION OF THE DRAWINGS

The present invention is illustrated by way of example and not limitation in the figures of the accompanying drawings in which like references indicate similar elements.

FIG. 1. An overview flow diagram presenting the modular components of the system and processes for communication between the users and the server and the screening process.

FIG. 2. A perspective view of an exemplary embodiment of the patch test kit to be delivered to the user. The outer layer is an opaque insulated envelope 101. Each patch test allergen panel is a single peel-off adhesive panel that carries one or more unique allergen(s) and is applied directly to the skin. The patch test allergen panel comprises multiple components 102,103,104,105, 106,107. A water-resistant outer layer secures the allergen panels for the duration of the application 102. Markings for allocation of the allergens, in this exemplary embodiment, a multicolored adhesive strip surrounds the allergen panel, arranged in a unique format for each panel according to the allergen composition to specify the allergens'location. Colored ornamentation also serves to calibrate the colors and facilitate the interpretation of skin reactions in different skin tones 103. The ornamentation may have various patterns, colors or orientation in different embodiments. Hypoallergenic, flexible pressure sensitive adhesive tape to carry multiple allergens directly placed on the skin 104. An occlusive or semi-occlusive polymer layer carries allergen film(s). In some embodiments this layer may be a flat film or a microneedle arrangement preloaded with allergens, or in the shape of a chamber for loading customized allergens. This layer may be used to optimize transcutaneous delivery of the allergens 105. Allergens are embedded in an inert excipient to form a thin film 106. A cover sheet is releasably attached to the adhesive panel, and allergens, to be removed before application on the skin 107. Each envelope is labeled with a user-specific QR code or another unique identifier on the outer package. A unique identifier or QR code is also used on each allergen panel specific for the individual patients and the number of allergen panels (most patients will require more than one panel; hence each panel will be numbered) 108.

FIGS. 3A-3B. A. front view of the allergen panel peeling the cover sheet. B. schematic cross-sectional side view of the allergen panel. A water-resistant outer layer 102 secures the allergen panels for the duration of the application on the skin, this layer will be peeled off after a certain application time. 103 is an example of markings presented as a multicolored adhesive strip surrounding the allergen panel, arranged in a unique format for each panel according to the allergen composition to help with allergen location, skin color calibration to normalize the skin reactions in different skin tones, and to adjust for body position. Adhesive allergen panel 104 is made of biocompatible hypoallergenic, flexible pressure-sensitive adhesive to carry one or more allergen(s), to be directly placed on the skin. An occlusive or semi-occlusive polymer 105 “allergen carrier” carries the allergen films and is used as backing for the allergen films to optimize cutaneous penetration. Allergen film(s) are allergens diluted in an inert excipient forming a thin film 106. A cover sheet is releasably attached to the adhesive panel and allergens 107.

FIGS. 4A-4C. Schema of exemplary embodiment using an adhesive patterned marking 109 along the borders of the allergen panel to assist with identifying specific allergen locations. FIGS. 4A, 4B, and 4C demonstrate the need for markings that allow allergen allocation when images are captured from various angles. Markings remain on the skin for from 1-7 days after allergens are removed to allow continued monitoring of skin reaction on each location until final interpretation of the patch test results. Patterns on the markers allow learning machine algorithms to accurately identify the allergen locations, even when follow up photos are captured from different angles. Various patterns, colors, shapes, orientation, and placement of markers can be used in different embodiments.

FIG. 5. An exemplary embodiment of a device to assist for allergen application and ensure proper attachment of the allergens to the skin.

FIG. 6. Flow diagram of embodiment of the system to assess probability of ACD in the present invention.

FIGS. 7A-7C. Schema of an exemplary process of allergen application, removal and sequential image capture. A. Schema of application of allergen panels by the user on healthy skin of trunk, arm, forearm, or thighs (allergens remain in contact with the skin for an average of two days. B. Removal of allergen panels by peeling off the waterproof top layer 102 and allergen panel 104, leaving the multicolored adhesive marking on the skin for continued monitoring of the reactions. C. Sequential images of the allergen application sites, one to four days after allergen removal. The multicolored adhesive tape is peeled off on the last day.

FIG. 8 Identification of the image corresponding to the allergen by a trained convolutional neural network (CNN). Final interpretation based by image feature extraction and integration with allergen skin-sensitizing potency and significance-prevalence index number (a composite measure of allergen prevalence and clinical relevance) to arrive at a probability distribution over International Contact Dermatitis Research Group scores. Colored and patterned biocompatible adhesive markings 110 can be used to allow accurate identification of the allergen location on follow up images and normalize reactions for various skin tones.

### DETAILED DESCRIPTION

The present invention offers systems, methods, and devices to facilitate access to care in patients with skin disorders which may include, but are not limited to, chronic eczema and other inflammatory skin disorders. More specifically, the present invention is directed to systems, methods, and devices for diagnosing and managing allergic contact dermatitis. In an exemplary embodiment of the present invention, the devices, systems, and methods allow for remote assessment of allergic contact dermatitis.

Prior patch tests require a patient to visit a clinic for the application of allergens to the skin and for interpretation of results and diagnosis. The systems, devices, and methods contemplated herein facilitate and simplify path-tests such that reduce patient burden, increase access, and allow for optional remote application, monitoring, and communication with an HCP. Improvements in the patch-test device allow for self-application and image capture.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

“Suitable conditions” shall have a meaning dependent on the context in which this term is used. That is, when used in connection with contacting an agent to a cell or tissue, this term shall mean conditions that permit an agent capable of doing so to enter a cell or tissue and perform its intended function. In one embodiment, the term “suitable conditions” as used herein means physiological conditions.

The terms “subject,” “individual,” and “patient” are used interchangeably herein to refer to a mammal being assessed. In an embodiment, the mammal is a human.

“In combination with”, “combination therapy” and “combination products” refer, in certain embodiments, to the concurrent administration to a patient of a first therapeutic (i.e., first therapeutic agent) and the compounds as used herein. When administered in combination, each component can be administered at the same time or sequentially in any order at different points in time. Thus, each component can be administered separately but sufficiently closely in time so as to provide the desired therapeutic effect. First therapeutic agents contemplated for use with the methods of the present invention include any other agent for use in the treatment of fibrosis. Examples of such therapeutic agents include but are not limited anti-fibrotic agents.

“Concomitant administration” of a known therapeutic agent with a pharmaceutical composition of the present invention means administration of the therapeutic agent and inhibitor agent at such time that both the known therapeutic agent and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention. Therapeutic agents contemplated for concomitant administration according to the methods of the present invention include any other agent for use in the treatment of fibrosis.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient ”means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

As used herein, the term “patch test” refers to the three-step process, which is the gold standard for in vivo assessment of delayed hypersensitivities associated with allergic contact dermatitis. This includes the following three steps: (1) direct application of allergens for a certain time, an average of two days using a biocompatible adhesive device for transcutaneous absorption of allergens (2) removal of allergens and continued monitoring of application sites for another two to four days (3) interpretation of results following the monitoring period.

The term “allergen panel” refers to the biocompatible adhesive sheet that carries one or more types of known allergens.

The term “test kit” refers to the device that contains allergen panel(s) with components required to complete and facilitate remote allergen application.

The term “allergen film” refers to a thin layer of standard doses of an allergen in an inert excipient.

The term “allergen carrier” refers to a thin nonpermeable or semipermeable sheet that on one side is attached to the allergen panel and on the side facing skin is covered with the allergen film. This layer serves as an intermediate layer between the allergen film and the adhesive panel.

The terms “allergen” and “hapten” may be used interchangeably.

Atopy patch test is an epicutaneous patch test with allergens, e.g. known to elicit IgE-mediated reactions, and used for the evaluation of eczematous skin reactions.

Atopy. Atopy is a predisposition to an immune response against diverse antigens and allergens leading to CD4⁺ Th2 differentiation and overproduction of immunoglobulin E (IgE). The clinical consequence is an increased propensity to hypersensitivity reactions. Allergic bronchial asthma and allergic rhinitis are frequent manifestations of atopy, followed by atopic dermatitis and food allergy. Other diseases described as atopic include allergic conjunctivitis, IgE-mediated drug allergy, urticaria and angioedema, and anaphylactic shock.

The pathophysiology of atopy characteristically demonstrates by mast cell activation. Antigen binding to IgE cross-links Fc epsilon RI proteins on mast cells. It activates protein tyrosine kinases (Lyn and Syk) that in turn cause activation of a MAP kinase cascade and a phosphatidylinositol-specific phospholipase C, which catalyzes the release of the following molecules: IP3 and DAG from membrane PIP2. Inositol trisphosphate (IP3) causes the release of intracellular calcium from the endoplasmic reticulum. DAG and calcium activate PKC that phosphorylates substrates such as myosin light chain molecule and thus leads to degradation and release of preformed mediators. MAP kinases and calcium react to activate the enzyme cytosolic phospholipase A2, which stimulates the synthesis of lipid mediators including PGD2, LTC4, LTD4, and LTE4. Ras/MAP kinases in the presence of calcium and PKC cause cytokine gene expression, which releases TNF and other cytokines (IL-4, IL-5, IL-6, IL-13 among others). Lipid mediators, cytokines and histamine cause an inflammatory response.

Atopy presents with a histopathologically characteristic wheal and flare reaction in the skin, which is in response to an allergen-stimulated release of mediators from mast cells, local blood vessels that dilate and become leaky to proteins and fluids, which produces local swelling and redness.

Atopic eczema (AE, atopic dermatitis, AE/dermatitis syndrome) is a clinically well-defined inflammatory, chronically relapsing, highly pruritic skin disease with a typically age-related distribution and morphology and a prevalence of 2% to 10% in the population. Elevated immunoglobulin E (IgE) production, especially against aeroallergens and food allergens, and/or altered unspecific reactivity are frequent findings in patients with AE and concomitant respiratory atopic diseases. As a multifactorial disease with a genetic background, AE has a large number of individually different trigger factors. The inflammatory infiltrate of AE lesions consists to a large proportion of CD4⁺ T helper (T_{H}) cells. High IgE production in patients with AE may be explained by an impaired balance of the T-cell populations T_{H}1 and T_{H}2, with a predominance of interleukin-4-and interleukin-13-producing T_{H}2 cells.

### Allergens

The patch test exposes the skin to a low dose of a known substance to elicit an allergic reaction in individuals who are already sensitized to this substance. The immune response triggered by these allergen(s) creates a localized inflammatory response on the skin at the exposure site (patch test application site). These reaction(s) to the allergens then informs the patient and HCP of which substances the patient is allergic to and needs to avoid using or contacting. Each substance will be prepared at concentrations recommended by medical literature and regulatory agencies that are only sufficient to illicit contact allergy in individuals that are already sensitized and do not induce sensitization. Inert excipients will be utilized to achieve the intended concentration and ensure the stability of the allergen compound. A thin layer of the allergen in the appropriate excipient will create a thin homogeneous film containing each allergen.

Examples of such allergens may include, without limitation, the following substances:
Nickel sulfate hexahydrate; Cobalt dichloride hexahydrate; p-tert-butylphenol formaldehyde resin; Epoxy resin; diglycidylether of bisphenol A; Carba mix including diphenyl guanidine; zincdibutyl dithiocarbamatee; zinc diethyldithiocarbamate; Quaternium-15; Wool alcohols; Potassium dichromate; Caine mix including benzocaine; tetracaine; dibucaine; Paraben mix including methylparaben; ethylparaben; propylparaben; butylparaben; benzyl paraben; Balsam of Peru resin; Ethylenediamine dihydrochloride; Black rubber mix including N-isopropyl-N′-phenyl paraphenylenediamine; N-cyclohexyl-N′-phenyl paraphenylenediamine; N, N′-diphenyl paraphenylenediamine; Methylchloroisothiazolinone (MCl) in combination with methylisothiazolinone (MI); Methyldibromo glutaronitrile; p-phenylenediamine; Mercapto mix including benzothiazole sulfenamide derivatives such as N-cyclohexyl benzo thiazyl-sulfenamide; dibenzothiazyl disulfide; morpholinyl mercapto benzothiazole); Thimerosol; Thiuram mix including tetramethyl thiuram monosulfide; tetramethyl thiuram disulfide; tetraethyl thiuram disulfide; dipentamethylenethiuram disulfide; Diazolidinyl urea; Quinoline mix (Clioquinol and clorquinaldol); Tixocortol-21-pivalate; Imidazolidinyl urea; Budesonide; Hydrocortisone-17-butyrate; Mercaptobenzothiazole; Parthenolide; Disperse blue 106; Bronopol; Fragrance mix, including cinnamaldehyde; hydroxycitronellal; cinnamyl alcohol; eugenol; isoeugenol; α-amyl cinnamaldehyde; oak moss; Colophony; Formaldehyde, etc. In some embodiments an allergen panel comprises at least the allergens present in a T.R.U.E. TEST® panel.

As is known to one skilled in the art, other substances, compounds, metals, or ions may also be considered as allergens and may be of interest for testing in the diagnosis of contact allergy. Contact irritants may be used in some embodiments.

Additional allergens may include, for example, occupational allergens causing IgE-mediated allergic asthma that includes animal products (cows, pigs, mice, dogs, cats, and horses), insect dusts (mealworms, storage mites, cockroaches, bees, and flies), plant products (dust, flours, grain and cotton dusts), fruits, seeds, leaves and pollens (castor beans, tobacco and weeping fig), vegetable, dusts, gums and extracts (western red, California redwood, and exotic woods), microbial agents (fungal allergens, alginates, protozoa, bacteria, and fungi), enzymes (papain, hog trypsin, pancreatic extracts, subtilisin, and pineapple bromelain), therapeutic agents (penicillins, tetracycline, cephalosporins, sulfonamides, and spiramycin), sterilizing agents (chloramides), inorganic chemicals (metal fumes and salts, aluminum, cobalt, fluoride, nickel, platinum, vanadium, and zinc) and organic chemicals (amines, anhydrides, and azodicarbonamide).

Samples that contain allergens may be obtained from plants (e.g. weeds, grasses, trees, pollens), animals (e.g., allergens found in the dander, urine, saliva, blood or other bodily fluid of mammals such as cat, dog, cow, pig, sheep, horse, rabbit, rat, guinea pig, mouse and gerbil), fungi/mold, insects (e.g., stinging insects such as bee, wasp, and hornet and chirnomidae (non-biting midges), as well as other insects such as the housefly, fruit fly, sheep blow fly, screw worm fly, grain weevil, silkworm, honeybee, non-biting midge larvae, bee moth larvae, mealworm, cockroach and larvae of Tenibrio molitor beetle; spiders and mites such as the house dust mite), rubbers (e.g. latex), metals, chemicals (e.g. drugs, protein detergent additives) and autoallergens and human autoallergens (e.g. Hom s 1, Hom s 2, Hom s 3, Hom s 4, Hom s 5) (see, Allergen Nomenclature: International Union of Immunological Societies Allergen Nomenclature Sub-Committee, List of allergens and Allergen Nomenclature: International Union of Immunological Societies Allergen Nomenclature Sub-Committee, List of isoallergens and variants).

To effectively deliver an allergen to the patient's skin, the allergen is placed in contact with the skin for a certain time period. The quantity and the concentration of the allergen that are exposed to skin is important to control and has been described. This can be controlled by adding a known amount of the allergen at a certain concentration and over a certain area 106 on a component of the patch test.

In some embodiments, the allergen is incorporated directly on the skin-contacting portion of component 104. This may be either on top of the allergen carrier or directly placed within the adhesive layer. Alternatively, the allergen is incorporated in regions of the adhesive side of component 104 where the adhesive material is absent. For example, the adhesive layer of component 104 may include regions that do not contain adhesive and are used to incorporate the allergen. In some embodiments these regions are in circles, squares, rectangles, or other shapes.

In some embodiments the allergen is incorporated into a separate layer of the patch-test, component referred to allergen carrier 105. This may be on one side of a material that is placed between the adhesive layer and the skin-contacting surface such that the allergen is in direct contact with the skin and is held in place by the surrounding adhesive.

It is important to deliver a controlled amount of allergen to the patient. The allergen may be incorporated into the patch-test or components of the patch-test. For example, a controlled amount of allergen may be coated onto a skin-contacting material, such as a natural or non-natural polymeric material. In certain embodiments the allergen is incorporated onto one side of the material. In certain embodiments the allergen is delivered to the material in a solution and the solution is then evaporated or dried. In certain embodiments the allergen is delivered from one or more solutions.

As known to one skilled in the art, allergens may be solubilized or delivered in different types of solutions, depending on a number of factors including the charge, hydrophobicity or hydrophilicity, or molecular weight of the allergen. Some chemicals, compounds, or metals may be soluble in polar solutions. Some chemicals, compounds, or metals may be soluble in non-polar solutions. Ideally, the solvent or solution that is the vehicle that delivers the allergen does not cause a biological response when in contact with the skin. In some embodiments, where possible, the vehicle is removed by methods such as evaporation or limited to where only a minimal amount of the vehicle is present. In other embodiments, the allergen is incorporated into the patch test through a matrix or polymeric compound that is biocompatible.

Polar solvents may include water, acetone, acetonitrile, dimethylformamide (DMF), dimelthylsulfoxide (DMSO), isopropanol, and methanol. Non-polar solvents may include alkanes (pentane, hexane, and heptane) and aromatics (benzene, toluene, and xylene). Other common nonpolar solvents include acetic acid, chloroform, diethyl ether, ethyl acetate, methylene chloride, and pyridine.

In certain embodiments the allergen is delivered in a polar or non-polar solvent that is then evaporated or dried. In other embodiments the allergen is delivered in a solvent or solution that is not evaporated or dried, such as a gel vehicle. In certain embodiments the allergen is delivered in one or more of polyvinylpyrrolidone (povidone), hydroxypropyl cellulose, and beta-cyclodextrin. In certain embodiments the pH of the gel is adjusted. In certain embodiments the pH is adjusted with acids or bases. Acids or bases may include one or more of those known to one skilled in the art. Exemplary acids may include hydrochloric acid, sulfuric acid, acetic acid, citrate, and the like. Exemplary bases may include tris, sodium carbonate, sodium bicarbonate, sodium hydroxide, and the like.

The composition and concentration of many allergens have been established and some are approved by regulatory agencies. These compositions and concentrations are known in the art and can be incorporated in the patch test described herein in accordance with established measures. In some embodiments, the composition and concentration of allergens may differ from those currently established and approved by regulatory agencies. The composition and concentration of each allergen may be determined from the established literature and known art and may be determined based on future scientific and clinical studies.

The dimension of the allergen contacting the skin is an important parameter as the dose of allergen per surface area in contact with the skin can influence the skin reaction. If the dose and/or surface area are too high or too low, the results may be misrepresented and misinterpreted. Additionally, the distance between allergens is an important parameter. If an allergen is placed in close proximity to another, the skin reaction may be misinterpreted and/or cross-contamination of the allergens could lead to an inaccurate interpretation.

In certain embodiments the dimensions of the allergen films 105 in contact with the skin are from about 25 mm² to about 500 mm², from about 25 mm² to 400 mm²; from about 25 mm² to 300 mm² ; from about 25 mm² to 400 mm².

The distance between allergens is important to prevent cross-contamination of allergens and to avoid misinterpreting test results if the skin reaction is too close to an adjacent allergen. In certain embodiments the distance between allergens 105, from the edge of one allergen to the closest edge of the next adjacent allergen, is from about 5 mm to about 100 mm, from about 5 mm to about 50 mm, from about 5 mm to about 25 mm, from about 5 mm to about 15 mm. from about 5 mm to about 10 mm.

It is important to prevent allergens from spreading from one specific site on the patient's skin to another. This may cause cross-contamination of allergens that would make it difficult to interpret or identify which allergen induces a reaction. As such, it is important to keep the allergen-containing region in place to prevent too much moisture, such as sweat or water, from leaking from one region to another. In certain embodiments, the allergen is kept in place on the skin surface and is protected from excessive moisture from skin through natural TEWL and sweat by the semi-permeable material used in component 105, which allows for adequate evaporation of sweat and prevents water from external sources such as a shower from penetrating into the skin-contacting and allergen-containing regions. In other certain embodiments the skin-contacting layer 104 and 102 contain porous regions in between allergens, that enhances breathability and reduces migration of moisture from one region to another. In some embodiments layers 102 and 104 are merged into one layer. In yet another embodiment, a barrier material separates the regions containing allergens. The barrier material may be composed of an absorbent material or a non-absorbent material.

### Method for Remote Screening and Diagnosis

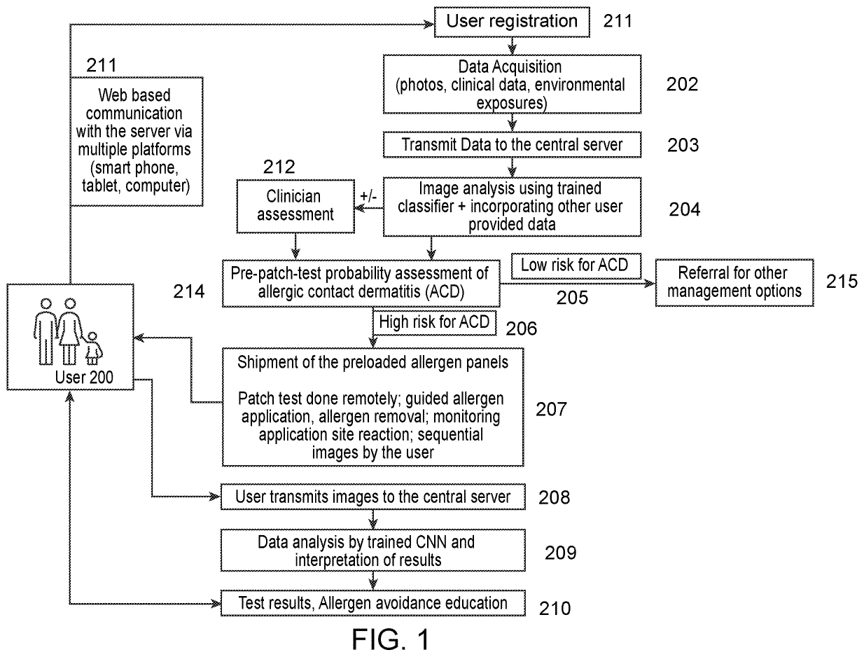

FIG. 1 provides an overview flow diagram presenting the modular components of the system and processes for communication between the users and the server and the screening process. In step 201 an individual user(s) 200 wishing to obtain information about their skin concerns, including rashes, lesions and areas of concern, or those wishing to have patch testing to screen for contact allergies will register on an online platform that is compliant with Health Insurance Portability and Accountability Act (HIPPA) and connected to a central server via internet 211, using an online application available on smartphones, tablets or computers and/or other a web-based devices. The registration provides identifiers for tracking analysis.

In step 202 additional information can be acquired through surveys to allow better assessment of the user's concerns by incorporating these data and image analysis. The requested information may include characteristics of the eruptions or lesions, such as duration, location, recurrence pattern, putative triggers, associated symptoms, skin contactants via occupational and environmental exposures, personal skin care product, medications. Surveys may include data on the users medical and surgical history as well as family history and relevant social history. In some embodiments, users upload images of written texts containing product ingredient lists, material safety data sheets, product safety data sheets, or voice messages, including the requested information. Digital images of the problematic areas of skin may be obtained. In some embodiments, trained algorithms are used to optimize image acquisition. The algorithm can be available to the user as an application compatible with the iPhone (Apple, Inc.), Windows Mobile, Google Android, and similar mobile phones and tablets to assist with image acquisition. A temporary marking of the skin in the form of a colored adhesive patch, QR code, or other forms are temporary marking may be used to help with the challenges of inadequate illuminations and various background skin colors. In some embodiments users are instructed to use optimized lighting and distance for high-quality images using a built-in camera on their smartphones, tablet, or digital camera and upload images to the server. In step 203 images and other data obtained are transmitted to a central server via internet using a mobile phone application or a website.

In the next step 204, a trained program in the central server performs initial image and text analysis to execute pre-processing algorithms for extracting relevant data, and a trained classifier distinguishes between inflammatory skin conditions including eczema and other conditions. A trained program can use additional survey data to assign a risk category ranging from unlikely to high for probability of allergic contact dermatitis. A password secured report is then transmitted to the user via the an interphase (such as a secure account accessed by a mobile phone application or a secure online website). Examples of classifiers that can be used in generating a probability score based on analysis of different features of interest can be found in U.S. Pat. No. 6,996,549 of Zhang, et al and U.S. Pat. No. 6,658,395 of Barnhill, each herein specifically incorporated by reference. The probability may be accompanied by additional explanation. In some embodiments additional information may be requested from the user in order to improve assessment and a clinician's input 212 will be incorporated to provide user with an initial assessment for pre-patch-test probability of allergic contact dermatitis, 214.

In step 205 users with a low likelihood of allergic contact dermatitis will be informed of the assessment and the central server will search a database to provide the user with referrals and resources with proper clinical qualifications for further assessments, 215. The present invention is not intended to provide a definitive diagnosis; it is only intended to serve as a readily accessible, low-cost preliminary screening tool to allow users to obtain an advisory indication of next steps in care and to facilitate formal clinical evaluation.

In step 206, an individual at high risk of ACD based on analysis of data and images individuals is identified. This data and information about the remote application of allergen panels and patch test procedure will be shared with users. In some embodiments users may choose to bypass steps 201, 202, 203, 204 and 214, and will proceed directly to patch testing.

In step 207 the preloaded allergen panels with use instructions are mailed to the user. In some embodiments a virtual visit with a trained medical professional can guide the users for application at home. In some embodiments the user is an individual performing the patch testing remotely at home or with the help of a trained medical professional visiting them at home or in another medical facility (such as a medical office, pharmacy, or a long-term care facility, etc).

In step 207 users are also guided through the patch test process as follows: preloaded allergen panels are applied on a healthy and clean part of the skin; the adhesive panels remain on skin for an average of two days. Duration might vary in different settings, certain circumstances or individual cases. In some embodiments a device such as a roller (shown in FIG. 5) is used to enforce secure attachment of the allergen panels to the skin. While there are minor variations worldwide in the patch test procedure, the most commonly recommended process is used to instruct users to apply the allergens for an average of two days. Users are instructed to obtain images of the skin in multiple steps. These image obtention steps may include some or all of the following steps: 1) prior to application of the allergens, as a baseline image of the skin where the allergens are to be applied; 2) after application, the skin where the adhesive allergen panels are placed; 3) the skin at the site, after removal of the allergen panels for the recommended period of time; 4) the skin at the site, after removal of the panels on subsequent 2 to 5 days. Users may obtain images on days after removal in the rare event that a delayed reaction occurs. In these cases, typically the skin markers/ornamentation are removed, while the algorithms for automatic pose matching can assist with identification, or users will be notified of resources with proper clinical qualification for further assessment.

In Step 208 all captured images are properly tagged by date and other identifiers. Unique markers in the form of adhesive-colored tapes or other markers can specify the location of allergens in the panel. All images are transmitted via internet to the central server on real time, or at a later date for analysis by trained CNN, for interpretation of the results 209.

In Step 210 users are notified of the test results. Those with an allergic reaction to a tested allergen will receive educational material regarding the tested material, and may received additional information regarding allergen avoidance. Referral resources for additional diagnostic workup and therapeutic management may be recommended to some users, by the central server and/or clinician input. In some embodiments users will have the option for ongoing interactive access to the central server.

### Device

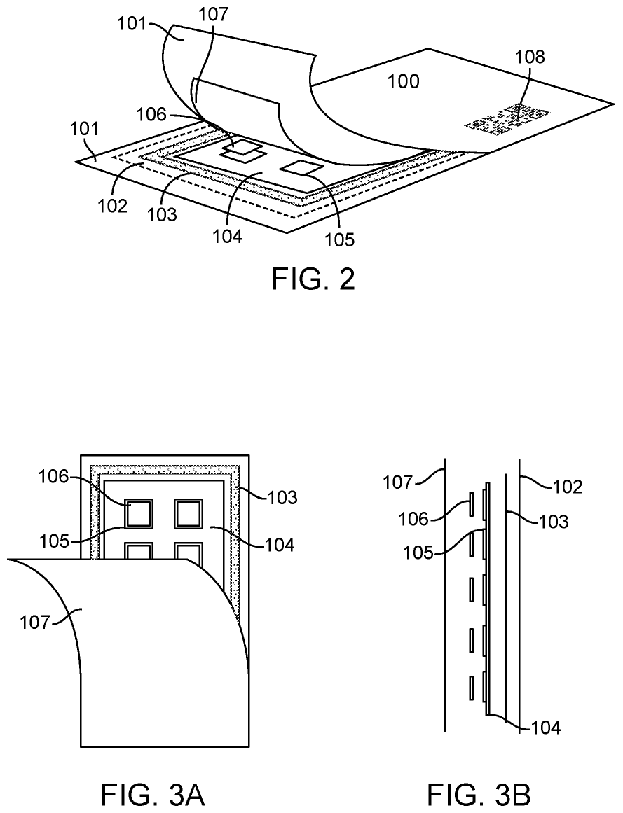

A Contact Allergen Panel is provided as a device, which can include components that facilitate the application of one or more allergens to the skin of a subject in need thereof. The device components can include one or more of a multilayer adhesive test panel, including a covering that may be waterproof or water-resistant, allergens, and one or more carriers for an allergen, as shown, for example, in FIG. 2, FIG. 3A and FIG. 3B.

In some embodiments, the device comprises one or more of the following components: a flexible water resistant or waterproof layer; a skin-contacting adhesive; and one or a panel of allergens (an irritant may also be used in some instances). Additional device features to facilitate use may include: a color normalization component; an allergen identification marker; an allergen-carrying component; a separation or barrier to prevent cross-contamination of allergens; an identification marker, such as for serial number and patient identification; and external packaging.

FIG. 2 provides a perspective view of an exemplary embodiment of the patch test device 100 to be delivered to the user. Each patch test allergen panel comprises a peel-off adhesive tape that carries one or more unique allergen(s) and is placed directly on the skin. The patch test allergen panel comprises multiple components 102, 103, 104, 105, 106, 107, 108 described below.

A principal goal of this invention is to facilitate the patch test process by introducing a new generation of allergen panels that can be readily applied, followed by a process for remotely monitoring allergen site reaction, and artificial intelligence augmented interpretation of the results. The allergen panel 100 is a ready-to-apply panel, in which allergens are incorporated in a thin, flexible gel 106 on top of a water-impermeable carrier sheet of low-risk polymers such as polyester polymers or plastic 105 and dried out to form a thin flexible film 106. The allergen film 106 and its carrier backing sheet 105 are mounted on a biocompatible pressure sensitive adhesive tape 104 that carries one or more allergen films 106.

The allergens are homogenized in one or more inert polymers that serve as a gel vehicle and are uniformly distributed to avoid uneven distribution in contact with skin. Allergen applications using film-forming polymers have been employed earlier in occlusive techniques and can be found, for example, in CA Pat. No. 1261746A by Fischer, incorporated herein by reference.

Allergen films 106 adhere well to the polymer backing sheets referred herein as allergen carrier film 105. The allergen films may contain hydrophilic vehicle gels to absorb natural moisture from skin, have low skin sensitizing potential and can vary to be suitable for the allergen they carry.

Various characteristics of the vehicle polymer sheets 105 are chosen to develop the proper vehicles. Suitable hydrophilic polymers may contain multiple polar structures, such as carboxyl and/or ester groups, amide groups, ether groups, completely alkylated polysaccharides, and alcoholic groups. Examples of polymers with high water absorption values which are suitable in this invention include, but are not limited to film-forming polysaccharides, polyvinyl alcohols, povidone, povidone with sodium bicarbonate, and more. The specific choice of vehicle gel for each allergen varies; for example, hydrophobic allergens, such as many fragrances, require vehicles that have both hydrophilic and lipophilic properties. The chemical nature of each allergen guides the proper formulation of each vehicle in accordance with the best-known method for producing the film-forming polymer.

The carrier film 105 is a non-irritating thin layer which is coated with the allergen film 106 on one side, and which contacts skin directly. It is mounted on the biocompatible pressure sensitive adhesive panel 104 on the side facing away from the skin. The carrier backing sheet 105 is preferably, but not necessarily water-impermeable, and can be made of various inert materials such as plastic, polyester or other hydrophobic polymers. The carrier sheets serve as a backing for the allergen films to ensure the proper occlusion of the allergen and optimize contact with skin. Components backing the allergen films may be semi-permeable materials. In some embodiments, the permeability of the material is measured by Moisture Vapor Transmission Rate (MVTR). In some embodiments, when a semi-permeable layer is used to cover the allergen films, the MVTR is calculated to be a less than average Trans Epidermal Water Loss (TEWL) to retain some moisture to facilitate allergen penetration, while preventing too much moisture retention.

Each allergen panel is composed of one or more pieces of uniformly distributed allergen films 106 and arranged and mounted on the biocompatible adhesive tape 104. The shape and arrangement of the allergen films may vary in various embodiments with proper distance between each allergen.

The most outer layer of the allergen panel is a water-resistant thin, flexible pressure sensitive adhesive 102 that adheres to the skin during the application period. This layer is made of a variety of water-resistant polymers such as polyurethane, or other polymers with similar properties. In some embodiments the allergen films and carriers are mounted directly to layer 102, eliminating the need for layer 104.

The surface of allergen films 106 is covered by a protective cover sheet 107 which is releasably attached to the adhesive panel. The cover sheet is removed immediately before application on the skin. Temporary marking is used to mark the location of each allergen after removal of the patches and for final interpretation. Examples of such marking includes a multicolored adhesive tape 109 and/or 103 surrounding the allergen panel arranged in various formation. These markings will remain on the skin for an additional period of time, typically from about 1 to about 7 days, until final set of images are captured for final interpretation of the patch test results. In different embodiments, various biocompatible temporary markings in various arrangements are employed.

The allergen panels are packaged in a laminated foil 101 appropriate for shipping and handling. In some embodiments the outer layer of the package and back of each allergen panel may be marked by a unique QR code 108 or another unique identifier per user and per panel.

Examples of adhesive sheets used in the allergen panel that can be used to carry the allergen test substances may include all or some of the following features: pressure-sensitive; biocompatible; conformable; breathable; and waterproof or water-resistant. The sheets are appropriate for short and medium-term wear on the skin. The adhesive carries the allergen films on the adhesive side, facing the skin, and may be covered by a releasably protective sheet that will be peeled off before applying on the skin.

In certain embodiments, device component 104 presented herein is an adhesive sheet that carries one or more allergen films and facilitates the application of the patch test, allowing for movement while holding the patch test and allergens in place. Component 104 may include a skin-contacting adhesive layer with an outer non-adhesive waterproof or water-resistant layer. To prevent any biological reaction with the skin, component 104 is made from a biocompatible material and adhesive to allow flexibility during movement. Component 104 is preferably made from a material with high conformability that can stretch and bend. To limit water from penetrating into the patch test, component 104 is preferably made of waterproof or water-resistant materials. Such biocompatible materials are known in the art and include natural and non-natural polymeric materials. In some embodiments, device component 104 has an adhesive side and a non-adhesive side. In some embodiments, device component 104 is composed of a latex-free hypoallergenic material. In some embodiments, device component 104 contains acrylic, polyethylene, polyurethane, polypropylene, polyolefin, silicone, rubber, or other materials as are known in the art.

In some embodiments, an additional adhesive layer 102 covers the allergen panel. Material(s) on the adhesive side of device component 102 is composed of a latex-free hypoallergenic pressure-sensitive adhesive. In some embodiments, the adhesive material allows for gentle removal of the patch test. In some embodiments, the adhesive material is composed of one or more synthetic rubber, acrylate, or silicone adhesive.

The properties of tape backings is important as the stiffness of the backing can influence how well an adhesive resists the peel forces the adhesive bond is exposed to. It is also important that the backing has high conformability so it can flex with the body's movement. Backing types may include paper, woven cloth, non-woven polyesters, foams, or polymeric films, such as polyurethane, polyethylene or polyester.

The breathability of the patch-test outer backing is another important parameter. Moisture from the skin (transepidermal water loss) facilitates transdermal delivery of the allergen(s) to the skin of the patient. If too much moisture is generated, there is a risk of spreading the allergens, which would result in dilution of the allergen or potentially cross-contaminating with one of the other nearby allergens. As such, in some embodiments, components backing the allergen films are semi-permeable materials. In some embodiments, the permeability of the material is measured by Moisture Vapor Transmission Rate (MVTR). In some embodiments, when a semi-permeable layer is used to cover the allergen films, the MVTR is calculated to be a less than average Trans epidermal water loss (TEWL) to retain some moisture to facilitate allergen penetration, while preventing too much moisture retention. Too much moisture can potentially cause irritation due to hyperhydration, increases the risk of allergen dispersing into adjacent areas of skin and adversely affect the diagnostic accuracy of the test.

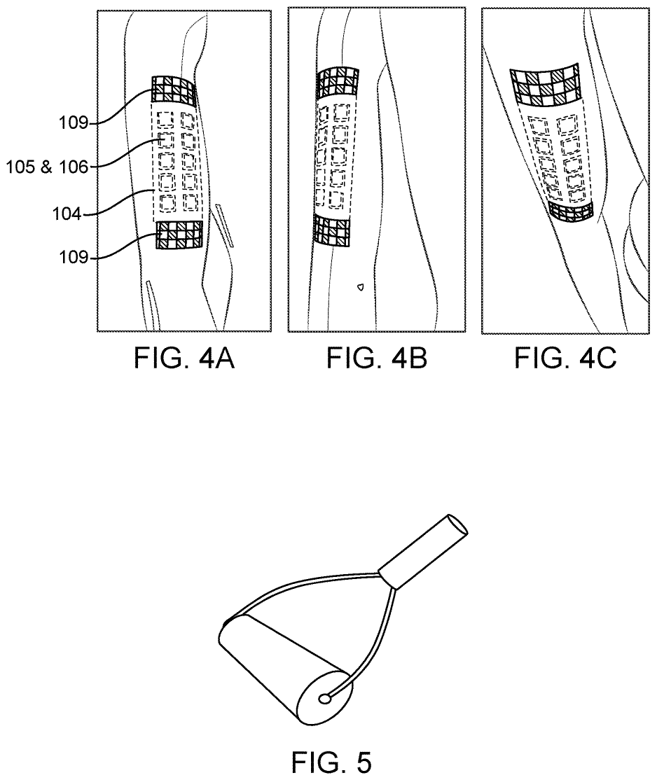

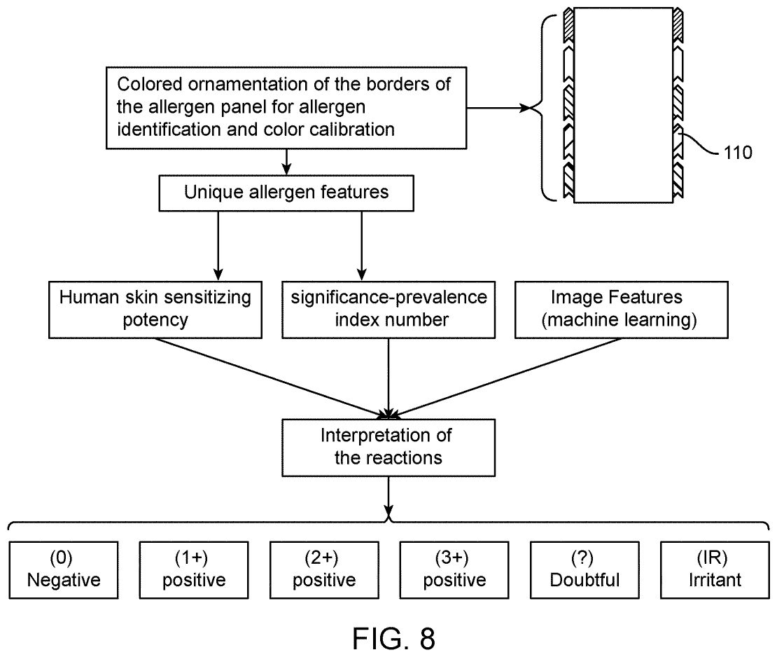

FIG. 4 provides a schema of exemplary embodiments using an adhesive patterned tape 109 used for marking along the borders of the allergen panel to assist with identifying specific allergen locations. FIGS. 4A, 4B, and 4C demonstrate how the angle of captures images may vary as the users will take sequential images during the patch test procedure. Due to the nature of delayed contact allergies the allergen application sites need to be monitored for a few days after allergen removal. Markings will remain on the skin for few days after allergens are removed to allow continued monitoring of skin reaction on each location until final interpretation of the patch test results. It is critical to identify allergen locations accurately, this invention uses various patterns on the markers that enables the machine learning algorithms to accurately identify the allergen locations even when follow up photos are captured from different angles. Various patterns, colors, shapes, orientation, and placement of markers may be used in different embodiments. Colored markers enable the learning machine algorithms to calibrate the skin reactions in different skin tones. Other examples of markings are presented on FIG. 8.

### Microneedle Array

In some embodiments the pressure sensitive 104 comprises a microneedle arrangement preloaded with allergens, in which embodiment an allergen film 105 is not required. This microneedle layer may be used to optimize transcutaneous delivery of the allergens. The use of microneedles in transdermal and intradermal delivery is advantageous as intracutaneous drug delivery or drug sampling can be accomplished by penetrating the outer layer of the skin without pain and bleeding. As used herein, the term “microneedles” refers to a plurality of elongated structures that are sufficiently long to penetrate through the stratum corneum skin layer into the epidermal or dermal or subcutaneous layer. In general, the microneedles are not so long as to penetrate into the dermal layer.

A microneedle array comprises a plurality of micro-projections, generally ranging from at least about 25 μm in length, at least about 100 μm in length, at least about 250 μm in length, at least about 500 μm in length, and not more than about 2000 μm in length, usually not more than about 1000 μm in length. In some embodiments the range is from about 100 to about 1000 μm in length; and some embodiments from about 250 to 750 μm in length. The microneedles are attached to the support, where the array is adhered to a patch of adhesive material that allows it to be fixed to the skin.

The microneedle arrays may comprise dissolving microneedles, e.g. of polyvinylpyrrolidone (PVP), which microneedles encapsulate allergens. In other embodiments a layer of the allergen coats the microneedles. The dose of allergens to be delivered will vary, and may range from at least about 1 ng/microneedle array, at least about 10 ng, at least about 0.1 μg, at least about 1 μg, at least about 10 μg or more in a single microneedle. The specific dose can be selected based on the allergen in question.

### Application of the Patch-Test

It is important that the patch-test is applied to the skin to effectively deliver the allergens to the skin. Having the patch-test flush and flat against the skin is ideal for optimal delivery of the allergen. It is important to limit gaps in the contact of the allergen with the skin. In certain embodiments an optional roller, shown in FIG. 5, is used to apply the patch-test to the skin. In other embodiments the patch-test is applied by hand. A roller or another device may be provided to assist with application of the pressure sensitive tape carrying the allergens.

### Methods and Ornaments for Digital Imaging

It is important to identify the location of each allergen to interpret the results and make an accurate diagnosis of which allergen the patient should avoid. The patch-test is commonly placed on the skin for a certain time period, and is then removed for a second time period. After the second time period, the reaction to an allergen is observed. Since the allergen component of the patch-test is not on the skin at the time of observation after the second time period, a method to identify the original location of the allergens is needed. As such there are multiple identification concepts described herein. In some embodiments the panel of one or more allergens is arranged specifically to meet personalized needs of individual patients.

In certain embodiments an adhesive identification marker remains on the skin after the allergen-containing portion of the patch-test has been removed. In another embodiment, a skin marker is used to identify the location and orientation of the allergens. The skin marker may be included in the skin-contacting adhesive layer such that the skin is marked when the patch-test is applied to the skin and does not require an additional step by the patient or HCP.

In another embodiment the patient takes a photograph of the patch-test when it is first applied to the skin and an image-processing algorithm is used to identify the location of each allergen after the patch-test is removed. For example, the algorithm may use an image registration feature that recognizes certain features on the patient's body and then maps these features to the original placement of the allergens.

Ideally, the patch-test can be used remotely such that direct interaction with a health care provider (HCP) in-person is not required. In some embodiments, device placement, removal, and the skin reaction response is managed by digital images captured with a phone, digital camera, and the like. Methods and instructions for image capture may be included in the patch-test kit and optional application that is used in combination with the patch-test kit. The quality of images obtained by a smart phone can be improved using an algorithm that captures the image with optimum resolution, distance, and lighting. In some embodiments, various ornamentations may be used to identify location of each allergen, orientation of the allergen panel as applied on the skin and to help with color calibration and improve resolution of the captured images.

Skin tone and color differ among people and may be modified by software within a digital camera or based on the lighting at the time an image was taken. It is therefore important to normalize the colors within a digital image to accurately assess the allergen response in different skin tones or colors.

In certain embodiments a color normalization feature is included in the patch-test kit. A color normalization component may include one or more reference colors that are captured within an image. An example includes but is not limited to a multicolored biocompatible adhesive patch that is applied to the skin for allergen mapping and to optimize normalization of the obtained images by color and size of the skin reactions to the patch test allergens, other forms of temporary marking may also be used to serve this purpose. This provides a reference that can be used for better visual representation of color or included in an algorithm to normalize the colors within an image.

In certain embodiments the color normalization feature is included as a part of an adhesive that is placed on the skin. In certain embodiments the color normalization adhesive is also used to identify the location of each allergen. In other embodiments the color normalization is not an adhesive, but is placed by the patient on or near the skin and is included in the image frame when a digital photograph is taken.

These systems and methods may be used to capture initial images of skin lesions and eruptions also for subsequent images as well as images from the patch test site reactions. In some embodiments automated body pose-matching algorithms may be used for continued monitoring of skin eruptions in an individual.

### Machine Learning Algorithms

Machine learning can aid in data interpretation and to facilitate automated diagnosis or to triage patient care. Machine learning has shown to be a promising in many fields including healthcare, as learning machines can be trained to solve problems known to those of ordinary skill in the art. Methods to use such knowledge for predicting and diagnosing allergic contact dermatitis can included in the patch-test. In some embodiments, these data and knowledge are collected and used for the diagnosis and management of other types of eczema and inflammatory skin disorders.

In certain embodiments a system for automated detection and interpretation of skin reactions to allergens by trained convolutional neural networks is used by incorporating the image features, integration with clinical history, allergen skin-sensitizing potency, and significance-prevalence index number (a composite measure of allergen prevalence and clinical relevance). In some embodiments, the data provided by machine learning algorithm(s) may be used by a HCP for further interpretation. In certain embodiments the data provided by machine learning algorithm(s) is used on its own for diagnosis and communication to the patient. In certain embodiments the data provided by machine learning algorithm(s) may be used to triage a patient for additional testing or to alert a HCP of a possible condition.

In some embodiments, a patient survey is combined with digital images for assessment. In certain embodiments, the digital images are taken with a digital camera or a Smart phone, such as the iPhone(R) (Apple, Inc.), Windows(R) Mobile, Google R Android, and similar mobile phones with PC-like functionality and cameras.

It is important for a patient and HCP to communicate via secure communication methods. If a patient and HCP meet in a remote or tele-health setting, communication needs to be through secure systems to protect private health information. As known to one skilled in the art, HIPAA compliant communication systems may be used for such communication.

In certain embodiments the data, such as digital images and patient survey data may be transmitted to a central server via internet (using an online application available on smartphones, or computers and/or a web-based platform (e.g. a website).

An algorithm that will discriminate robustly between allergen responses, and controls is useful for this purpose. In such a process, the raw photographic data can be manipulated according to an algorithm, where the algorithm has been pre-defined by a training set of data.

A classification process may use any one of a variety of statistical analytic methods to manipulate the data and provide for classification of the sample. Examples of useful methods include linear discriminant analysis, recursive feature elimination, a prediction analysis of images, a logistic regression, a CART algorithm, a FlexTree algorithm, a LART algorithm, a random forest algorithm, a MART algorithm, machine learning algorithms; neural networks, principle components analysis, recursive partitioning, predictive algorithms, Bayesian networks, etc. Two sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome. Several of these methods are based on the “R” software, developed at Stanford University, which provides a statistical framework that is continuously being improved and updated in an ongoing basis.

In the generation of such a model, a dataset comprising positive and negative control images may be used as a training set.

Classification can be made according to predictive modeling methods that set a threshold for determining the probability that a sample belongs to a given class, i.e. responsive, non-responsive, etc. The probability preferably is at least 50%, or at least 60% or at least 70% or at least 80% or higher. The predictive ability of a model may be evaluated according to its ability to provide a quality metric of a particular value, or range of values.

Also provided are databases and computer systems for analysis. Databases can typically comprise distribution pattern information from various conditions, such as responses of cells to a variety of treatments. The results and databases thereof may be provided in a variety of media to facilitate their use.

“Media” can refer to a manufacture that contains the response information; and methods of analysis as described above. The databases and comparative algorithms can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information provided herein. The minimum hardware of the computer-based systems of the present invention comprises a central processing unit (CPU), input means, output means, and data storage means. A skilled artisan can readily appreciate that any one of the currently available computer-based system are suitable for use in analysis. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression repertoire.

The data analysis may be implemented in hardware or software, or a combination of both. In one embodiment, a machine-readable storage medium is provided, the medium comprising a data storage material encoded with machine readable data which, when using a machine programmed with instructions for using said data, is capable of displaying a any of the datasets and data comparisons of this invention. Such data may be used for a variety of purposes, such as drug discovery, analysis of interactions between cellular components, and the like. In some embodiments, the analysis is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language may be a compiled or interpreted language. Each such computer program can be stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems. One format for an output tests datasets possessing varying degrees of similarity to a trusted repertoire. Such presentation provides a skilled artisan with a ranking of similarities and identifies the degree of similarity contained in the test repertoire.

Further provided herein is a method of storing and/or transmitting, via computer, data collected by the methods disclosed herein. Any computer or computer accessory including, but not limited to software and storage devices, can be utilized to practice the present invention. Data can be input into a computer by a user either directly or indirectly. Additionally, the user input can be linked to a computer, such that the data is transferred to a computer and/or computer-compatible storage device. Data can be stored on a computer or suitable storage device (e.g., CD). Data can also be sent from a computer to another computer or data collection point via methods well known in the art. Thus, data collected by the methods described herein can be collected at any point or geographical location and sent to any other geographical location.

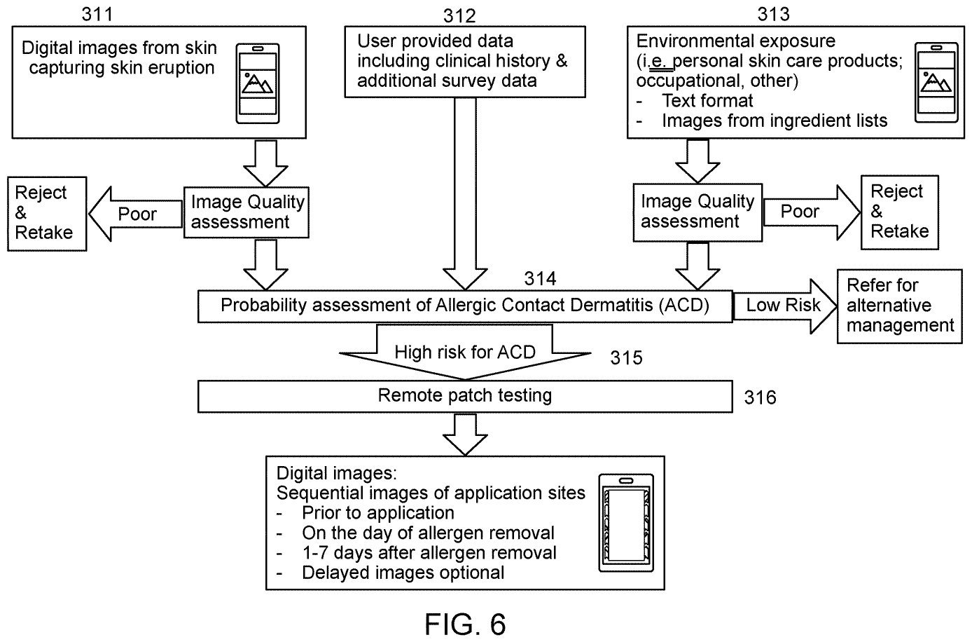

FIG. 6 provides a flow diagram of embodiment of the system to assess probability of ACD in the present invention. Data acquisition including digital images 311, clinical history based on patient surveys 312 and additional information on environmental exposures and skin contactants 313 will be transmitted to the central server via internet for analysis via a trained learning machine. In some embodiments additional information may be collected and clinician input may be added. Based on the collective data users with low likelihood of allergic contact dermatitis will be informed of the assessment and the central server will search a database to provide the user with referrals and resources with proper clinical qualifications for further assessments 314 and users who are deemed to benefit from screening for contact allergy by patch testing 315 will be notified the assessment and next steps and guided through the remote patch test procedure 316.

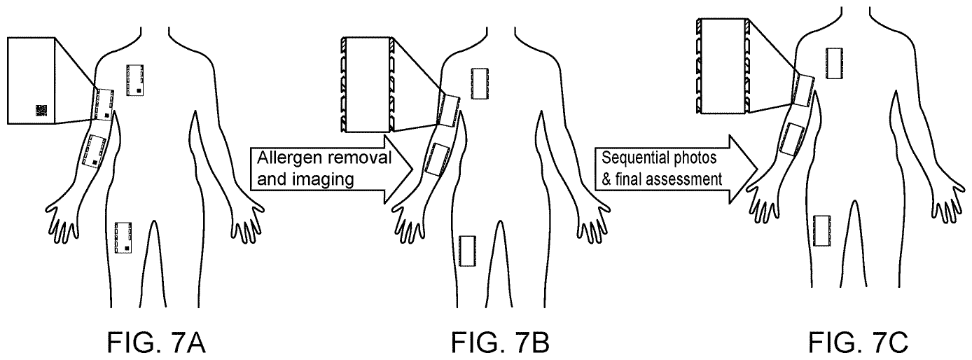

FIG. 7 provides a schema of an exemplary process of allergen application, removal and sequential image capture. FIG. 7A is a schema of application of allergen panels by the user on healthy skin of trunk, arm, forearm, or thighs (allergens will remain in contact with skin for certain amount of time an average of two days, e.g. from about 24 to about 72 hours, from about 18 hours to about 60 hours, from about 24 hours to about 48 hours, and may be around about 48 hours. The duration of application may vary in individual users. FIG. 7B. Removal of allergen panels by peeling of the waterproof top layer 102 and allergen panel 104 in certain embodiments, where the allergen panel also serves as the waterproof covering layer only the allergen panel will be removed. The markings used to allocate allergens 110 will remain in place for an additional period of time typically one to four days, until final interpretation of patch test results. Herein an example of a multicolored adhesive linear tape along the sides of the allergen panel is used as a marker 110; in various embodiments different shapes, arrangements, materials, and colors may be used for marking the skin for continued monitoring of the reactions. FIG. 7C shows sequential images of the allergen application sites, at various intervals, typically one to four days after allergen removal. Skin markings will be removed on the final day. In some embodiments a digital application is available to users that assists with capturing the images using body posing algorithms to help with continued monitoring of the allergen application sites.

FIG. 8 shows identification of the image corresponding to the allergen by a trained convolutional neural network (CNN). Final interpretation is based by image feature extraction and integration with allergen skin-sensitizing potency and significance-prevalence index number (SPIN), a composite measure of allergen prevalence and clinical relevance, to arrive at a probability distribution over International Contact Dermatitis Research Group scores from negative reaction to weak positive (1+) strong positive (2+ and 3+), doubtful and Irritant reaction. Utilizing a composite measure by incorporating SPIN score and allergen skin-sensitizing potency allows better interpretation of doubtful skin reactions.

While the present invention is not intended to provide a definitive diagnosis; it allows screening for contact allergy to common allergens. Once an allergen is identified, users are informed of the findings, will be provided by educational material about the allergen(s) identified and will be provided resources to further manage their contact allergies. This system and method provides a readily accessible and cost effective resource that allows users to obtain an advisory indication of next steps in care and to facilitate formal clinical evaluation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

### REFERENCES

1. Silverberg J I, Simpson E L. Associations of Childhood Eczema Severity: A US Population-Based Study. Dermatitis. 2014;25(3):107-114. doi:10.1097/DER.0000000000000034
2. Chiesa Fuxench Z C, Block J K, Boguniewicz M, et al. Atopic Dermatitis in America Study: A Cross-Sectional Study Examining the Prevalence and Disease Burden of Atopic Dermatitis in the US Adult Population. J Invest Dermatol. 2019;139(3):583-590. doi:10.1016/j.jid.2018.08.028
3. Lim H W, Collins S A B, Resneck J S, et al. Contribution of health care factors to the burden of skin disease in the United States. J Am Acad Dermatol. 2017;76(6):1151-1160.e21. doi:10.1016/j.jaad.2017.03.006
4. Boonchai W, Charoenpipatsin N, Winayanuwattikun W, Phaitoonwattanakij S, Sukakul T. Assessment of the quality of life (QoL) of patients with dermatitis and the impact of patch testing on QoL: A study of 519 patients diagnosed with dermatitis. Contact Dermatitis. n/a(n/a). doi:10.1111/cod.13535
5. Rajagopalan R, Anderson R. Impact of patch testing on dermatology-specific quality of life in patients with allergic contact dermatitis. Am J Contact Dermat Off J Am Contact Dermat Soc. 1997;8(4):215-221.
6. el Samahy M H, el-Kerdani T. Value of patch testing in atopic dermatitis. Am J Contact Dermat Off J Am Contact Dermat Soc. 1997;8(3):154-157.
7. Owen J L, Vakharia P P, Silverberg J I. The Role and Diagnosis of Allergic Contact Dermatitis in Patients with Atopic Dermatitis. Am J Clin Dermatol. 2018;19(3):293-302. doi:10.1007/s40257-017-0340-7
8. Malajian D, Belsito D V. Cutaneous delayed-type hypersensitivity in patients with atopic dermatitis. J Am Acad Dermatol. 2013;69(2):232-237. doi:10.1016/j.jaad.2013.03.012
9. A. Chiang, S. Li and G. Honari. Cost benefits of patch testing in patients with dermatitis. Contact Dermatitis.
10. Johansen J D, Aalto-Korte K, Agner T, et al. European Society of Contact Dermatitis guideline for diagnostic patch testing—recommendations on best practice. Contact Dermatitis. 2015;73(4):195-221. doi:10.1111/cod.12432
11. Fischer T I, Maibach H I. The thin layer rapid use epicutaneous test (TRUE-test), a new patch test method with high accuracy. Br J Dermatol. 1985;112(1):63-68. doi:10.1111/j.1365-2133.1985.tb02292.x
12. Smith Y, Czerning R. Medical Imaging Method and System. Published online Oct. 23, 2008. Accessed Jun. 12, 2022. https://patents.google.com/patent/US20080260218/en?oq=Harald+SCHNIDAR
13. Neubauer A, SCHNIDAR H. EP29760 13A1, Methods for assessing erythema. Published online Jan. 27, 2016.
14. Basketter D A, Alépée N, Ashikaga T, et al. Categorization of chemicals according to their relative human skin sensitizing potency. Dermat Contact Atopic Occup Drug. 2014;25(1):11-21. doi:10.1097/DER.0000000000000003
15. DeKoven J G, Warshaw E M, Zug K A, et al. North American Contact Dermatitis Group Patch Test Results: 2015-2016. Dermat Contact Atopic Occup Drug. 2018;29(6):297-309. doi:10.1097/DER.0000000000000417
16. Svedman C, Isaksson M, Björk J, Mowitz M, Bruze M. “Calibration” of our patch test reading technique is necessary. Contact Dermatitis. 2012;66(4):180-187. doi:10.1111/j.1600-0536.2011.02044.x
17. Hamann D, Hamann C R, Hamann C. A contemporary Fischer-Maibach investigation: variations in patch test delivery systems and implications for standardization. Dermat Contact Atopic Occup Drug. 2013;24(6):302-312. doi:10.1097/DER.0b013e31829f28ac
18. Zhang H, Carls G, Barnhill S D. Computer-aided image analysis. Published online Feb. 7, 2006.
19. Barnhill S D. Enhancing knowledge discovery using support vector machines in a distributed network environment. Published online Dec. 5, 2000. Accessed Jul. 15, 2022. https://patents.google.com/patent/US6157921A/en?oq=6%2c157%2c921
20. Tanno L K, Darlenski R, Sánchez-Garcia S, et al. International survey on skin patch test procedures, attitudes and interpretation. World Allergy Organ J. 2016;9:8. doi:10.1186/s40413-016-0098-z
21. Fischer T I. CA1261746A Hypersensitivity test means.
22. Mark H F, Seidel A. Encyclopedia of Polymer Science and Technology. Wiley; 2014.

1. A method for remote assessment of delayed-type contact allergies by transcutaneous application of contact allergens, comprising:
(a) registering a user;
(b) providing the user with a Contact Allergen Panel;
(c) guiding the user to perform allergen panel application at a test site, removal of the allergen panel, and image acquisition of the test site;
(d) receiving transmission of site images from the user;
(e) analyzing the image data to make a determination of responsiveness to allergens provided in the contact allergen panel.

2. The method of claim 1, wherein the Contact Allergen Panel comprises a pattern that specifies the location of each allergen present in the panel.

3. The method of claim 1, wherein the Contact Allergen Panel comprises a pattern that provides a point of reference for monitoring the skin reactions after removal of the allergens.

4. The method of claim 1, wherein the Contact Allergen Panel comprises a pattern that provides color calibration to normalize skin reactions according to skin tone.

5. The method of claim 1, wherein determination of responsiveness is performed with a machine learning algorithm.

6. The method of claim 5, wherein the machine learning comprises trained convolutional neural networks based on image feature extraction and integration with allergen skin-sensitizing potency and significance-prevalence index number to arrive at a probability distribution over International Contact Dermatitis Research Group scores.

7. The method of claim 5, further comprising a clinician evaluation of results.

8. The method of claim 1, comprising a secure internet interface for communications with the user.

9. The method of claim 8, wherein one or more of software, hardware and communication tools are provided to the user for a secure internet interface.

10. The method of claim 1, comprising a pre-patch test probability assessment of allergic contact dermatitis (ACD) for the user, wherein a user with a high probability of ACD is provided with a contact allergen panel and instructions for use.

11. A Contact Allergen Panel for use in the method of claim 1, comprising:
a flexible water-resistant or waterproof layer;
a skin-contacting adhesive;
one or a plurality of allergens.

12. The allergen panel of claim 11, further comprising an empty chamber suitable for additional allergens.

13. The allergen panel of claim 11, further comprising a color normalization component

14. The allergen panel of claim 11, further comprising an allergen identification marker.

15. The allergen panel of claim 11, wherein allergens are provided in a film adhered to the skin contacting adhesive.

16. The allergen panel of claim 11, wherein allergens are provided in a microneedle array adhered to the skin contacting adhesive.

17. The allergen panel of claim 11, further comprising separation or a barrier to prevent cross-contamination of allergens

18. The allergen panel of claim 11, further comprising an identification marker.

19. The allergen panel of claim 11, further comprising external packaging.

20. The allergen panel of claim 11, wherein the allergens comprise one or more of Nickel sulfate hexahydrate; Cobalt dichloride hexahydrate; p-tert-butylphenol formaldehyde resin; Epoxy resin; diglycidylether of bisphenol A; Carba mix including diphenyl guanidine; zincdibutyl dithiocarbamatee; zinc diethyldithiocarbamate; Quaternium-15; Wool alcohols; Potassium dichromate; Caine mix including benzocaine; tetracaine; dibucaine; Paraben mix including methylparaben; ethylparaben; propylparaben; butylparaben; benzyl paraben; Balsam of Peru resin; Ethylenediamine dihydrochloride; Black rubber mix including N-isopropyl-N′-phenyl paraphenylenediamine; N-cyclohexyl-N′-phenyl paraphenylenediamine; N, N′-diphenyl paraphenylenediamine; Methylchloroisothiazolinone (MCl) in combination with methylisothiazolinone (MI); Methyldibromo glutaronitrile; p-phenylenediamine; Mercapto mix including benzothiazole sulfenamide derivatives such as N-cyclohexyl benzo thiazyl-sulfenamide; dibenzothiazyl disulfide; morpholinyl mercapto benzothiazole); Thimerosol; Thiuram mix including tetramethyl thiuram monosulfide; tetramethyl thiuram disulfide; tetraethyl thiuram disulfide; dipentamethylenethiuram disulfide; Diazolidinyl urea; Quinoline mix (Clioquinol and clorquinaldol); Tixocortol-21-pivalate; Imidazolidinyl urea; Budesonide; Hydrocortisone-17-butyrate; Mercaptobenzothiazole; Parthenolide; Disperse blue 106; Bronopol; Fragrance mix, including cinnamaldehyde; hydroxycitronellal; cinnamyl alcohol; eugenol; isoeugenol; α-amyl cinnamaldehyde; oak moss; Colophony; and Formaldeh
